(19) United States
(12) Patent Application Publication
(10) Pub. No.: US 2020/0262794 A1
(43) Pub. Date: Aug. 20, 2020

(54) GLYCOLATE OXIDASE INHIBITORS AND METHODS OF USE FOR THE TREATMENT OF KIDNEY STONES

(71) Applicants: Wake Forest University Health Sciences, Winston-Salem, NC (US); UAB Research Foundation, Birmingham, AL (US)

(72) Inventors: W. Todd Lowther, Pfattown, NC (US); Ross P. Holmes, Birmingham, AL (US); Daniel Yohannes, Winston-Salem, NC (US)

(21) Appl. No.: 15/781,571

(22) PCT Filed: Dec. 7, 2016
(86) PCT No.: PCT/US2016/065300

§ 371 Date: Jun. 5, 2018

Related U.S. Application Data
(60) Provisional application No. 62/263,938, filed on Dec. 7, 2015.

(51) Int. Cl. C07D 233/90; C07D 409/12; C07D 409/06; C07D 249/04; C07D 231/18; C07D 207/456; A61P 13/04

(52) U.S. Cl. CPC C07D 233/90; C07D 409/12; C07D 409/06; A61K 9/0053; C07D 231/18; C07D 207/456; A61P 13/04; C07D 249/04

(57) ABSTRACT

Provided herein are compounds of Formula I and Formula II, and compositions comprising the same, as well as methods of use thereof for treating kidney stones (e.g., inhibiting the formation of oxalate kidney stones; treating primary hyperoxaluria), inhibiting the production of glyoxylate and/or oxalate, and/or inhibiting glycolate oxidase (GO).

### RELATED APPLICATIONS

This application claims the benefit of U.S. Provisional Patent Application Ser. No. 62/263,938, filed Dec. 7, 2015, the disclosure of which is incorporated by reference herein in its entirety.

### GOVERNMENT FUNDING

This invention was made with government support under grant numbers DK083527 and DK073732 awarded by National Institutes of Health. The United States government has certain rights in the invention.

### BACKGROUND

Kidney stones affect approximately 1 in 11 individuals in the United States. The 2012 National Health and Nutrition and Examination Survey (NHANES), part of the Urological Diseases in America Project, reported that the overall prevalence of kidney stones was 8.8% (10.6% and 7.1% for men and women, respectively) (Jiang et al., Am J Physiol Gastrointest Liver Physiol 302, G637-643, 2012). This study and others attest to the significant increase in stone cases in general, but especially in individuals with obesity, diabetes, and following bariatric surgery (Jiang et al., supra; Knight et al., Am J Nephrol 25, 171-175, 2005). The direct and indirect costs associated with kidney stone treatment (i.e., nephrocalcinosis) are significant (Knight et al., Kidney Int 70, 1929-1934, 2006).

Individuals with Primary Hyperoxaluria (PH) have mutations in a variety of genes involved in glyoxylate and hydroxyproline (Hyp) metabolism that result in a significant increase in oxalate production and deposition of calcium oxalate stones, the most common type of stones for all stone formers. The treatments for these individuals range from a combined kidney-liver transplant to a life-long use of potassium citrate, increased fluid intake and dietary restriction of oxalate (Riedel et al., PLoS One 6, e26021, 2011; Knight et al., Am J Physiol-Renal 302, F688-693, 2012). Treatments for the removal of stones currently include shock-wave lithotripsy, ureteroscopic stone removal, and percutaneous nephrolithotomy (Riedel et al., supra). However, the recurrence of stones following the available procedures is over 50%.

Kidney stones are also a significant problem in veterinary medicine. Pets such as dogs and cats can develop stones that lead to painful urination and/or a life-threatening blockage.

Considering that the current treatments only address symptoms, novel treatments to prevent the formation of stones in PH and other idiopathic stone formers are greatly needed.

### SUMMARY

Provided herein are compounds of Formula I:

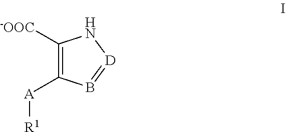

wherein:

A is CH₂ or S;

B is CH or N;

D is CH or N; and

R¹ is aryl or heteroaryl, wherein said aryl or heteroaryl has two aromatic rings, which rings are fused or directly adjoining,

or a pharmaceutically acceptable salt or prodrug thereof.

In some embodiments, A is CH₂. In some embodiments, A is S. In some embodiments, B is CH. In some embodiments, B is N. In some embodiments, D is CH. In some embodiments, D is N.

In some embodiments, R¹ is benzothiophene or biphenyl.

In some embodiments, R¹ is selected from the group consisting of:

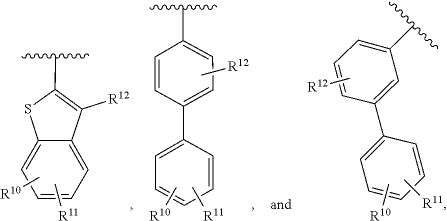

wherein R¹⁰, R¹¹ and R¹² are each independently selected from the group consisting of: H, alkyl, halo and haloalkyl,

or a pharmaceutically acceptable salt or prodrug thereof.

Also provided are compounds of Formula II:

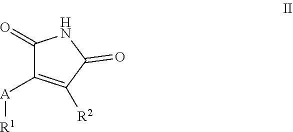

wherein:

A is CH₂ or S;

R¹ is aryl or heteroaryl, wherein said aryl or heteroaryl has two aromatic rings, which rings are fused or directly adjoining; and

R² is H or OH,

or a pharmaceutically acceptable salt or prodrug thereof.

In some embodiments, A is CH₂. In some embodiments, A is S. In some embodiments, R² is H. In some embodiments, R² is OH.

In some embodiments, R¹ is benzothiophene or biphenyl.

In some embodiments, R¹ is selected from the group consisting of:

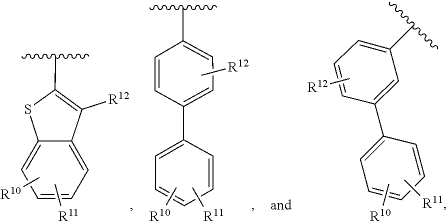

wherein R¹⁰, R¹¹ and R¹² are each independently selected from the group consisting of: H, alkyl, halo and haloalkyl,

or a pharmaceutically acceptable salt or prodrug thereof.

Also provided are pharmaceutical compositions comprising a compound, pharmaceutically acceptable salt or prodrug as taught herein. In some embodiments, the composition is formulated for oral administration. In some embodiments, the composition is a food product formulation.

Further provided are methods of treating kidney stones (e.g., inhibiting the formation of oxalate kidney stones; treating primary hyperoxaluria), comprising: administering to a subject in need thereof a therapeutically effective amount of a compound, pharmaceutically acceptable salt or prodrug as taught herein.

Still further provided are methods of inhibiting the production of glyoxylate and/or oxalate, and/or inhibiting glycolate oxidase (GO), in a subject in need thereof, comprising: administering to said subject a therapeutically effective amount of a compound, pharmaceutically acceptable salt or prodrug as taught herein.

Also provided is the use of the compound, pharmaceutically acceptable salt or prodrug as taught herein, or a pharmaceutical composition as taught herein, for treating kidney stones (e.g., inhibiting the formation of oxalate kidney stones; treating primary hyperoxaluria), inhibiting the production of glyoxylate and/or oxalate, and/or inhibiting glycolate oxidase (GO), in a human or non-human animal subject in need thereof.

Further provided is the use of a compound, pharmaceutically acceptable salt or prodrug as taught herein in the preparation of a medicament for treating kidney stones (e.g., inhibiting the formation of oxalate kidney stones; treating primary hyperoxaluria), inhibiting the production of glyoxylate and/or oxalate, and/or inhibiting glycolate oxidase (GO), in a human or non-human animal subject in need thereof.

### BRIEF DESCRIPTION OF THE DRAWINGS

FIG. 1 presents a schematic of the metabolism of 4-hydroxyproline, glycolate and glyoxylate within a hepatocyte. Four mitochondrial enzymes are responsible for Hyp breakdown: hydroxyproline dehydrogenase (HYPDH), Δ¹-pyrroline-5-carboxylate dehydrogenase (1P5CDH), aspartate aminotransferase (AspAT), and 4-hydroxy-2-oxoglutarate aldolase (HOGA). A variety of enzymes, including alanine-glyoxylate aminotransferase (AGT), D-amino acid oxidase (DAO), glyoxylate reductase (GR), and lactate dehydrogenase (LDH), can act on the glyoxylate produced from HOG cleavage. AGT, GR, and HOGA are mutated within primary hyperoxaluria patients (PH type 1, 2, and 3, respectively). Glycolate oxidase (GO) can readily convert glycolate back into glyoxylate within the peroxisome; a feature that is particularly problematic for PH2 patients.

### DETAILED DESCRIPTION

Provided herein are methods of treatment for controlling or inhibiting the formation of kidney stones comprising administering to a subject in need thereof an inhibitor of glycolate oxidase (GO), as well as compounds and compositions useful for the same.

The disclosures of all patent references cited herein are hereby incorporated by reference to the extent they are consistent with the disclosure set forth herein. As used herein in the description of the invention and the appended claims, the singular forms “a,” “an” and “the” are intended to include the plural forms as well, unless the context clearly indicates otherwise.

“Subject” or “patient” as used herein are generally mammalian subjects, including both human subjects and non-human mammalian subjects (e.g., dog, cat, horse, etc.) for research or veterinary purposes. Subjects may be male or female and may be of any suitable age, including neonate, infant, juvenile, adolescent, adult, and geriatric subjects.

“Treat” as used herein refers to any type of treatment that imparts a benefit to a subject, particularly slowing or inhibiting the formation of glyoxylate and/or oxalate, decreasing urinary oxalate, slowing or inhibiting the formation of calcium oxalate stones in the kidneys and/or urinary tract (kidneys, ureters, bladder, and urethra), and/or the deposition of calcium oxalate in other tissues such as the heart. For example, the treatment may reduce the size of and/or decrease the number of such stones, inhibit or slow the growth of such stones or calcium oxalate deposition in tissues such as the heart, alleviate symptoms of such stones or deposition, etc. Treatment may also include prophylactic treatment of a subject deemed to be at risk of kidney stone formation (e.g., after bariatric surgery and recurrent idiopathic “stone formers”).

“Kidney stones” are hard deposits of minerals that form a stone or crystal aggregation, which may result in damage or failure of the kidney and/or urinary tract function. Most kidney stones are calcium stones, usually in the form of calcium oxalate.

“Oxalate” or “oxalic acid” is a dianion of the formula C₂O₄²⁻ produced by the body and also commonly ingested in the diet. Oxalate can combine with calcium in the kidneys or urinary tract to &um calcium oxalate, which is the main component of most kidney stones.

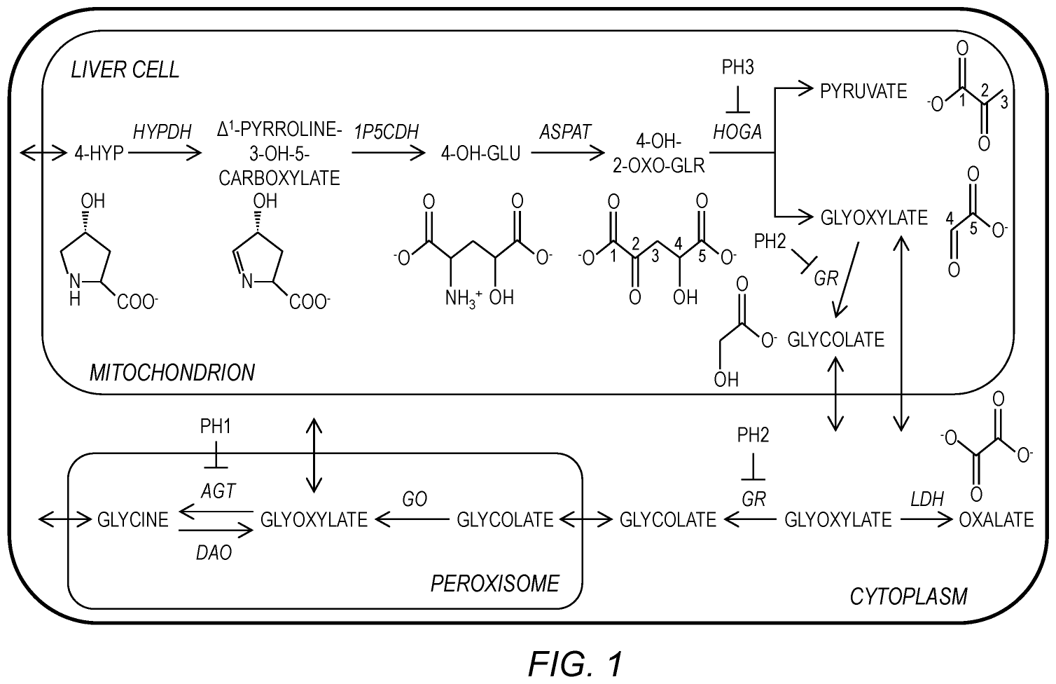

“Glyoxylate” is a precursor of oxalate, as shown in FIG. 1.

“Glycolate oxidase” or “GO” is an enzyme that catalyzes the oxidation of glycolate. Multiple GO isoforms exist, such as GO1 (predominantly in liver) and GO2 (located in kidney and liver) (Jones et al. J Biol Chem 275, 12590-12597, 2000). GO1 catalyzes the FMN-dependent oxidation of glycolate to glyoxylate, and glyoxylate to oxalate, although the latter occurs with a 100-fold lower kcat/Km value (Murray et al. Biochemistry 47, 2439-2449, 2008).

“Primary hyperoxaluria” is a condition characterized by the overproduction of oxalate and/or defective production or function of one or more enzymes that regulate the levels of oxalate in the body. Sufferers of Type 1 primary hyperoxaluria have a defect or shortage of the alanine:glyoxylate aminotransferase enzyme (AGT). Type 2 primary hyperoxaluria sufferers have a defect or shortage of the glyoxylate reductase enzyme (GR). Type 3 primary hyperoxaluria sufferers have a defect or shortage of the 4-hydroxy-2-oxoglutarate aldolase (HOGA).

“Hydroxyproline” or “Hyp” has the structure:

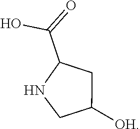

Hydroxyproline is produced in the body primarily from endogenous collagen turnover (Miyata et al., Proc Nati Acad Sci USA 111, 14406-14411, 2014). Using a unique metabolic tracer, ¹³C₅,¹⁵N-Hyp (all five carbons isotope and nitrogen atom labeled), it was determined that the level of Hyp turnover could be as high as 6-7 g/day (Riedel et al., Biochim Biophys Acta 1822, 1544-1552, 2012). Less than 5 mg of free Hyp is excreted in urine each day, indicating that most of the Hyp is metabolized (Belostotsky et al., J Mol Med (Berl) 90, 1497-1504, 2012). This significant metabolic load could contribute up to 25% of the endogenous oxalate produced (Phang et al., (2001) Disorders of proline and hydroxyproline metabolism. in The Metabolic and Molecular Bases of Inherited Disease (Scriver, C. R., Beaudet, A. L., Sly, W. S., Vallee, D., Childs, B., Kinzler, K. W., and Vogelstein, B. eds.), McGraw-Hill, New York. pp 1821-1838). The biological reason why Hyp metabolism occurs is not clear, although it does enable some pyruvate to feed back into other pathways.

Hyp is metabolized primarily in the mitochondria of the liver and renal cortical tissue (Kivirikko, Int Rev Connect Tissue Res 5, 93-163, 1970; Atlante et al., Biochem Biophys Res Commun 202, 58-64, 1994; Monico et al., Clin J Am Soc Nepthrol 6, 2289-2295, 2011; Wold et al., J Food Sc 64, 377-383, 1999). Diet can also be a source of collagen. For example, a quarter pound hamburger rich in gristle could contain as much as 6 grams of collagen, yielding 780 mg of Hyp (Khan et al., J Urol 184, 1189-1196, 2010). In fact, dietary Hyp can significantly increase oxalate production in humans and lead to hyperoxaluria in mouse and rat models (Khan et al., Kidney Int 70, 914-923, 2006; Valle et al., J Clin Invest 64, 1365-1370, 1979; Adams et al., Annu Rev Biochem 49, 1005-1061, 1980).

FIG. 1 presents the Hyp catabolic pathway and the metabolism of glyoxylate and glycolate. The Hyp pathway involves four enzymatic reactions (Miyata et al., Proc Natl Acad Sci USA 111, 14406-14411, 2014; Efron et al., New Engl J Med 272, 1299-1309, 1965; Pelkonen et al., New Engl J Med 283, 451-456, 1970). The first step of the pathway is the flavin FAD-dependent oxidation of Hyp to Δ¹-pyrroline-3-hydroxy-5-carboxylate (3-OH-P5C) by HYPDH. The 3-OH-P5C intermediate is converted to 4-hydroxy-glutamate (4-OH-Glu) by 1P5C dehydrogenase (1P5CDH), an NAD+-dependent enzyme shared with the proline degradation pathway (Efron et al., supra). Aspartate aminotransferase (AspAT) utilizes oxaloacetate to convert 4-OH-Glu to 4-hydroxy-2-oxoglutarate (HOG). HOG is then cleaved by the unique HOG aldolase (HOGA) into two fragments, glyoxylate and pyruvate. The glyoxylate can then be converted to glycolate and glycine via glyoxylate reductase (GR) and alanine:glyoxylate aminotransferase (AGT), respectively. Glycolate can be converted back into glyoyxlate by glycolate oxidase (GO).

AGT, GR, and HOGA are mutated within primary hyperoxaluria patients (PH type 1, 2, and 3, respectively). For PH1 and PH2 patients, the glyoxylate produced from Hyp could exacerbate the already high levels of glyoxylate, and increase oxalate production via the lactate dehydrogenase (LDH). For PH3 patients, HOGA is inactivated, leading to a buildup of HOG (Riedel et al., Biochim Biophys Acta 1822, 1544-1552, 2012; Belostotsky et al., J Mol Med (Berl) 90, 1497-1504, 2012). Recent studies identified that HOG can inhibit GR, potentially leading to a PH2-like phenotype (Riedel et al., Biochim Biophys Acta 1822, 1544-1552, 2012). In contrast, glycolic aciduria, caused by deficiencies in GO, is not associated with any overt consequences, and glycolate can be excreted (Frishberg et al. J Med Genet 51, 526-529, 2014).

Thus, and without wishing to be bound by theory, inhibition of GO by a small molecule inhibitor that targets the enzyme active site is not expected to lead to any adverse side effects, and will block the formation of glyoxylate and oxalate from glycolate for all PH patient types. Inhibition of GO is also expected to help idiopathic stone formers and other individuals with high urinary oxalate levels, such as those that have undergone gastric bypass surgery. For the latter, there is a significant increase in stone formation that may benefit from prophylactic treatment post surgery. While the exact origins of the oxalate in these patients has not been determined, inhibition of HYPDH will decrease glyoxylate and oxalate levels, which will ultimately reduce the glyoxylate and oxalate burden in them.

### 1. Active Compounds

Active compounds as described herein can be prepared in accordance with known procedures or variations thereof that will be apparent to those skilled in the art.

As will be appreciated by those of skill in the art, the active compounds of the various formulas disclosed herein may contain chiral centers, e.g., asymmetric carbon atoms, and the present disclosure is inclusive of both: (i) racemic mixtures of the active compounds, and (ii) enantiomeric forms of the active compounds. The resolution of racemates into enantiomeric forms can be done in accordance with known procedures in the art. For example, the racemate may be converted with an optically active reagent into a diastereomeric pair, and the diastereomeric pair subsequently separated into the enantiomeric forms.

Also included in active compounds disclosed herein are tautomers (e.g., tautomers of triazole and/or pyrazole) and rotamers.

As described herein, certain groups or portions of the compounds of the invention may optionally be substituted with one or more substituents, such as those illustrated generally herein. In general, the term “substituted” refers to the replacement of hydrogen in a given structure with a substituent. Unless otherwise indicated, a substituted group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable compounds. “Stable” as used herein refers to a chemically feasible compound that is not substantially altered when kept at a temperature of 40 ° C. or less, in the absence of moisture or other chemically reactive conditions, for at least a week.

As used herein in the accompanying chemical structures, “H” refers to a hydrogen atom. “C” refers to a carbon atom. “N” refers to a nitrogen atom. “S” refers to a sulfur atom.

The term “hydroxy,” as used herein, refers to a group —OH.

“Carbonyl” is a group having a carbon atom double-bonded to an oxygen atom (C═O).

“Carboxy” as used herein refers to a group —COOH or —COO⁻.

“Amine” or “amino” refers to a group —NH₂.

“Halo” is a halogen group selected from the group consisting of fluoro (—F), choro (—Cl), bromo (—Br), and iodo (—I). “Haloalkyl” is a halogen group connected to the parent compound by an alkyl group.

“Alkyl,” as used herein, refers to a saturated straight or branched chain, or cyclic hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, cyclopropyl, cyclobutyl, cyclopentyl, cyclohexyl, and the like. “Lower alkyl” as used herein, is a subset of alkyl and refers to a straight or branched chain hydrocarbon group containing from 1 to 4 carbon atoms. Representative examples of lower alkyl include, but are not limited to, methyl, ethyl, n-propyl, iso-propyl, n-butyl, iso-butyl, tert-butyl, cyclopropyl, cyclobutyl, and the like. The alkyl groups may be optionally substituted with one or more suitable substituents, such as halo, hydroxy, carboxy, amine, etc.

“Aryl,” as used herein, refers to a monocyclic carbocyclic ring system or a bicyclic carbocyclic fused or directly adjoining ring system having one or more aromatic rings. Examples include, but are not limited to, phenyl, indanyl, indenyl, tetrahydronaphthyl, and the like. As noted, in some embodiments, the aryl has two aromatic rings, which rings are fused or directly adjoining. Examples include, but are not limited to, biphenyl, napthyl, azulenyl, etc. The aryl may be optionally substituted with one or more suitable substituents, such as alkyl, halo, hydroxy, carboxy, amine, etc.

“Heteroaryl,” as used herein, refers to a monovalent aromatic group having a single ring or two fused or directly adjoining rings and containing in at least one of the rings at least one heteroatom (typically 1 to 3) independently selected from nitrogen, oxygen and sulfur. Examples include, but are not limited to, pyrrole, imidazole, thiazole, oxazole, furan, thiophene, triazole, pyrazole, isoxazole, isothiazole, pyridine, pyrazine, pyridazine, pyrimidine, triazine, and the like. As noted, in some embodiments, the heteroaryl has two aromatic rings, which rings are fused or directly adjoining. Examples include, but are not limited to, benzothiophene, benzofuran, indole, benzoimidazole, benzthiazole, quinoline, isoquinoline, quinazoline, quinoxaline, phenyl-pyrrole, phenyl-thiophene, etc. The heteroaryl may be optionally substituted with one or more suitable substituents, such as alkyl, halo, hydroxy, carboxy, amine, etc.

A “pharmaceutically acceptable salt” is a salt that retains the biological effectiveness of the free acids or bases of a specified compound and that is not biologically or otherwise undesirable. Examples of pharmaceutically acceptable salts may include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, gamma-hydroxybutyrates, glycollates, tartrates, methane-sulfonates, propanesulfonates, naphthalene-1-sulfonates, naphthalene-2-sulfonates, and mandelates.

A “prodrug” is a compound that is converted under physiological conditions or by solvolysis or metabolically to a compound that is pharmaceutically active. A thorough discussion is provided in T. Higuchi and V. Stella, Prodrugs as Novel delivery Systems, Vol. 14 of the A.C.S. Symposium Series and in Edward B. Roche, ed., Bioreversible Carriers in Drug Design, American Pharmaceutical Association and Pergamon Press, 1987, both of which are incorporated by reference herein in their entirety. See also Huttunen et al., “Prodrugs—from Serendipity to Rational Design,” Pharmacological Reviews 63(3):750-771 (2011), which is incorporated by reference herein. Example prodrugs include, but are not limited to, the addition of/conversion to phosphate(s), amino acid esters, amino acid amides, sugar derivatives, alkyl or aryl esters, etc., at an —OH, —SH, —NH or —COOH group of the parent active compound.

Provided herein as active compounds according to some embodiments are compounds of Formula I:

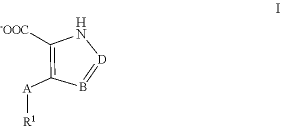

wherein:

A is CH₂ or S;

B is CH or N;

D is CH or N; and

R¹ is aryl or heteroaryl, wherein said aryl or heteroaryl has two aromatic rings, which rings are fused or directly adjoining,

or a pharmaceutically acceptable salt or prodrug thereof.

In some embodiments of Formula I, A is CH₂. In some embodiments, A is S. In some embodiments, B is CH. In some embodiments, B is N. In some embodiments, D is CH. In some embodiments, D is N.

In some embodiments of Formula I, R¹ is benzothiophene or biphenyl.

In some embodiments of Formula I, R¹ is selected from the group consisting of:

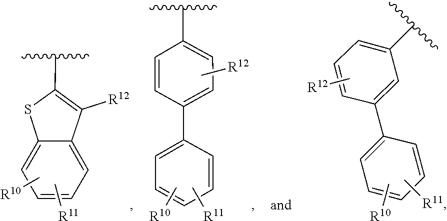

wherein R¹⁰, R¹¹ and R¹² are each independently selected from the group consisting of: H, alkyl, halo and haloalkyl.

Also provided are GO inhibitor compounds of Formula II:

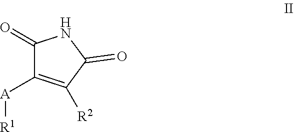

wherein:

A is CH₂ or S;

R¹ is aryl or heteroaryl, wherein said aryl or heteroaryl has two aromatic rings, which rings are fused or directly adjoining; and

R² is H or OH,

or a pharmaceutically acceptable salt or prodrug thereof.

In some embodiments of Formula II, A is CH₂. In some embodiments, A is S.

In some embodiments of Formula II, R¹ is benzothiophene or biphenyl.

In some embodiments of Formula II, R¹ is selected from the group consisting of:

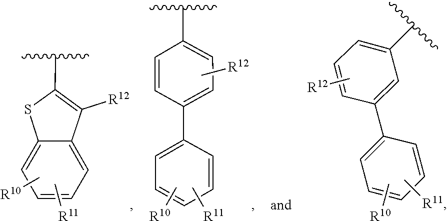

wherein R¹⁰, R¹¹ and R¹² are each independently selected from the group consisting of: H, alkyl, halo and haloalkyl.

### 2. Formulations

The active compounds described herein may be formulated for administration in a pharmaceutical carrier in accordance with known techniques. See, e.g., Remington, The Science and Practice of Pharmacy (9^{th} Ed. 1995). In the manufacture of a pharmaceutical formulation according to the invention, the active compound (including the physiologically acceptable salts or prodrugs thereof) is typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5% to 95% or 99% by weight of the active agent. One or more active agents may be incorporated in the formulations of the invention, which may be prepared by any of the well-known techniques of pharmacy comprising admixing the components, optionally including one or more accessory ingredients.

The pharmaceutical compositions may also contain other additives, such as pH-adjusting additives. In particular, useful pH-adjusting agents include acids, such as hydrochloric acid, bases and/or buffers, such as sodium lactate, sodium acetate, sodium phosphate, sodium citrate, sodium borate, or sodium gluconate. Further, the compositions may contain preservatives. Useful preservatives include methylparaben, propylparaben, benzoic acid and benzyl alcohol.

The formulations may comprise nanoparticles, such as biodegradable polymers and/or liposome-forming material, for encapsulation and/or delivery of the active agent(s). See, e.g., WO 2014/201312 to Wang et al.; Cho and Jung, “Supramolecular Complexation of Carbohydrates for the Bioavailability Enhancement of Poorly Soluble Drugs,” Molecules 20:19620-19646, 2015; Nogueira et al., “Design of liposomal formulations for cell targeting,” Colloids Surf B Biointerfaces 136:514-526, 2015. In some embodiments, liver-targeting nanoparticles may be used for specific delivery of active agent(s) acting at the liver. See, e.g., U.S. 2015/0150994 to Hahn et al.; U.S. 2008/0138394 to Kim et al. In some embodiments, kidney-targeting nanoparticles may be used for specific delivery of active agent(s) acting at the kidney. See, e.g., U.S. Pat. No. 8,318,199 to Kim et al.; U.S. 2012/0196807 to Nakamura et al.

In some embodiments, the active agent(s) may be provided in a controlled-release or sustained-release formulation. See, e.g., Grinyo and Petruzzelli, “Once-daily LCP-Tacro MeltDose tacrolimus for the prophylaxis of organ rejection in kidney and liver transplantations,” Expert Review of Clinical Immunology 10(12):1567-1579, 2014 (Erratum: Expert Review of Clinical Immunology 11(4):547, 2015).

Formulations of the invention may include those suitable for oral, buccal (sub-lingual), parenteral (e.g., subcutaneous, intramuscular, intradermal, or intravenous), topical (i.e., both skin and mucosal surfaces, including airway surfaces) and transdermal administration, although the most suitable route in any given case will depend on the nature and severity of the condition being treated and on the nature of the particular active compound being used.

Formulations suitable for oral administration may be presented in discrete units, such as capsules, cachets, lozenges, or tablets, each containing a predetermined amount of the active compound(s); as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for oral administration also include food product formulations, such as a nutritional bar or an animal feed (e.g., pet food such as dog or cat food). Food product formulations may include one or more of carbohydrates such as wheat, corn rice, barley or oats, dairy products such as milk, oils such as canola oil or soybean oil, flavorants such as sugar or syrup, coloring, chocolate, preservatives, etc. Pet food formulations, in particular, may include meat, poultry, fish or other animal-derived components such as eggs.

Formulations suitable for buccal (sub-lingual) administration include lozenges comprising the active compound in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions, which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, in one aspect of the present invention, there is provided an injectable, stable, sterile composition comprising an active compound(s) in a unit dosage form in a sealed container. The active compound(s) may be provided in the form of a lyophilizate which is capable of being reconstituted with a suitable pharmaceutically acceptable carrier to form a liquid composition suitable for injection thereof into a subject.

When the active compound(s) is substantially water-insoluble, a sufficient amount of emulsifying agent which is physiologically acceptable may be employed in sufficient quantity to emulsify the compound or salt in an aqueous carrier. One such useful emulsifying agent is phosphatidyl choline.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound. Suitable formulations comprise citrate or bis\tris buffer (pH 6) or ethanol/water and contain from 0.1 to 0.2M active ingredient.

The unit dosage foini typically comprises from about 1 mg, 5 mg, 10 mg, 100 mg, 250 mg, 500 mg, 1 gram, 5 grams, 10 grams, or any ranges therein, of the active compound(s), depending on the subject being treated (e.g., human or non-human mammalian subject). In some embodiments, the unit dosage form is in the range of 500 mg to 10 grams, keeping in mind that a good portion of the active compound(s) may not be absorbed upon administration (e.g., oral adminstration).

The present invention is explained in greater detail in the following non-limiting examples.

### EXAMPLES

Example 1. Glycolate oxidase (GO) inhibitor design. Based on crystal structures of human GO1 with CCPST and CDST as well as other biochemical data, GO inhibitors are designed to exploit one or more of the following interactions:

(1) force W110 to “flip” out of the active site, causing loop 4 to become disordered;

(2) protonated nitrogen at position 3 directly interacts with the catalytic residue His260;

(3) carboxylate interaction with one or both of two conserved Arg residues.

Example 2. Example GO inhibitors. With the above considerations in mind, the following compounds are designed as GO inhibitors.

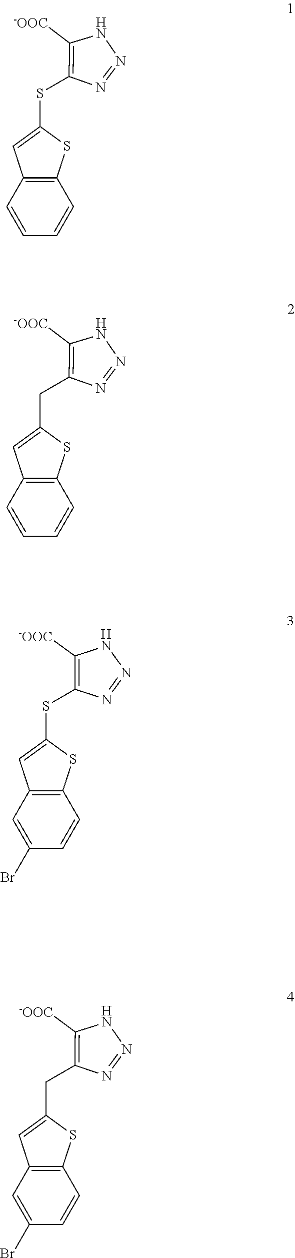

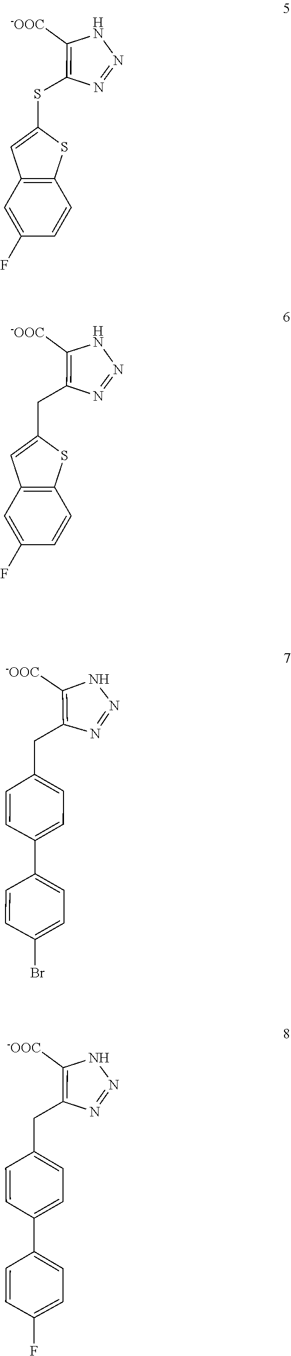

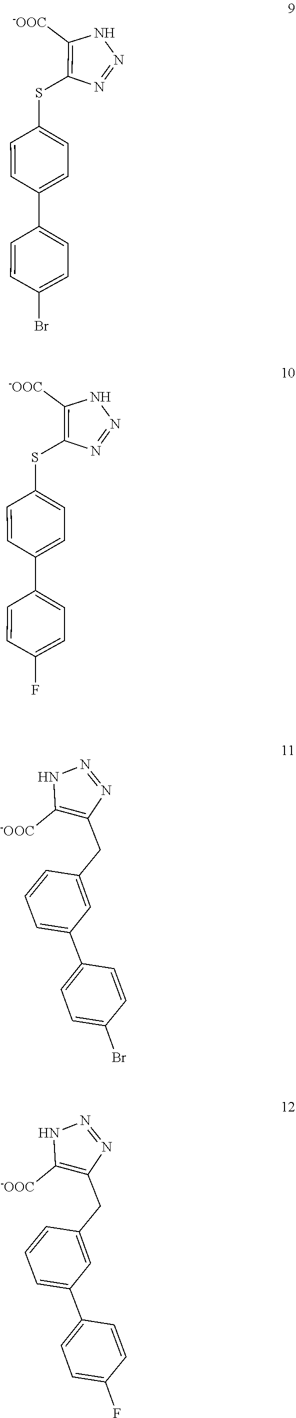

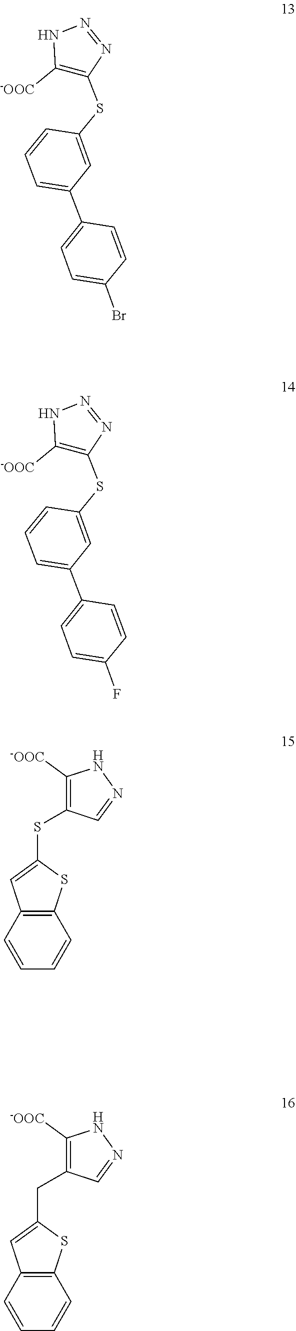

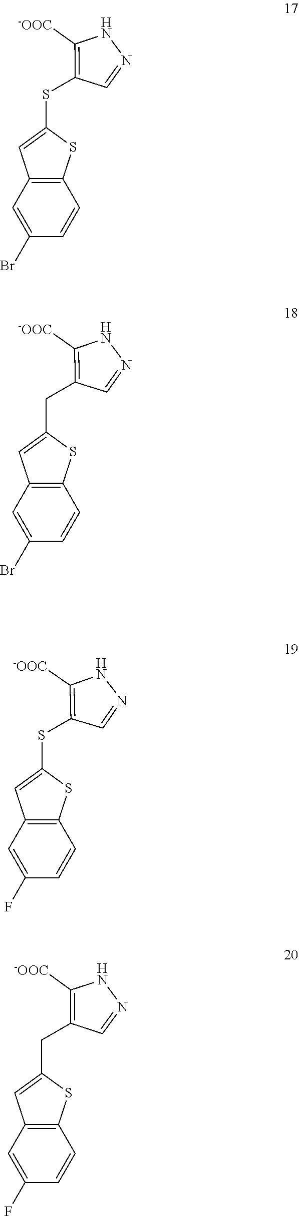

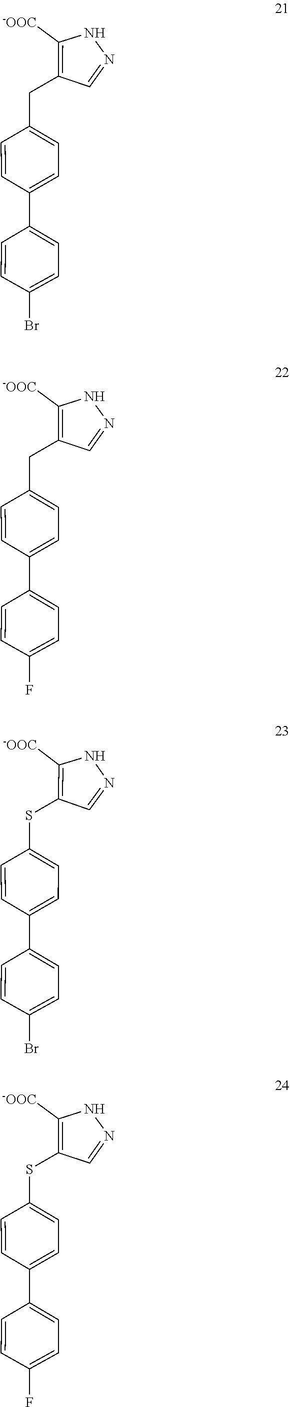

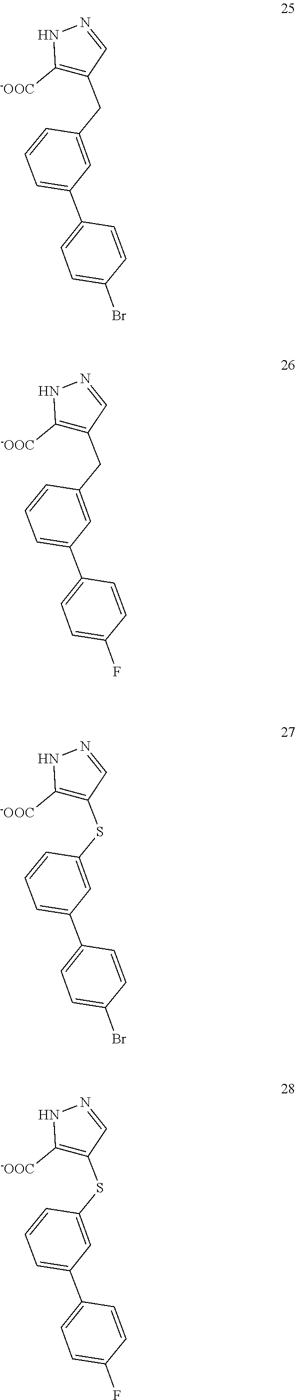

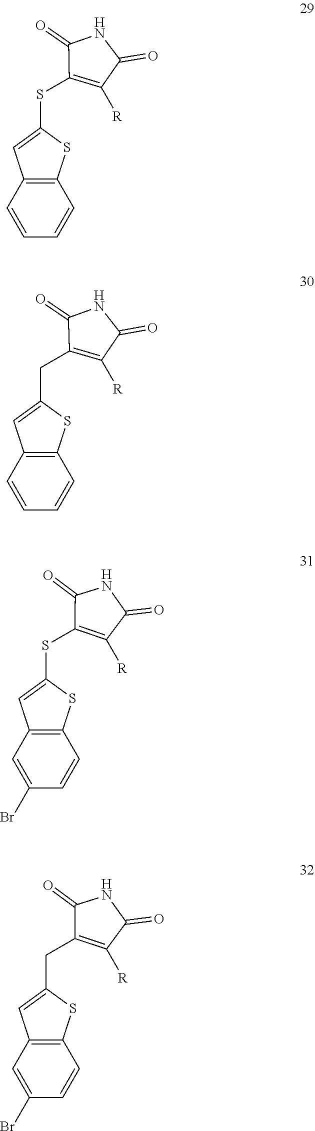

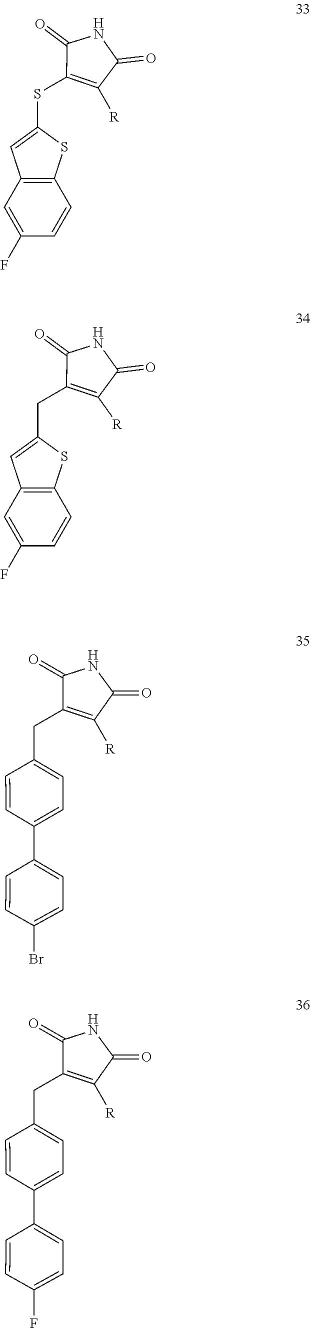

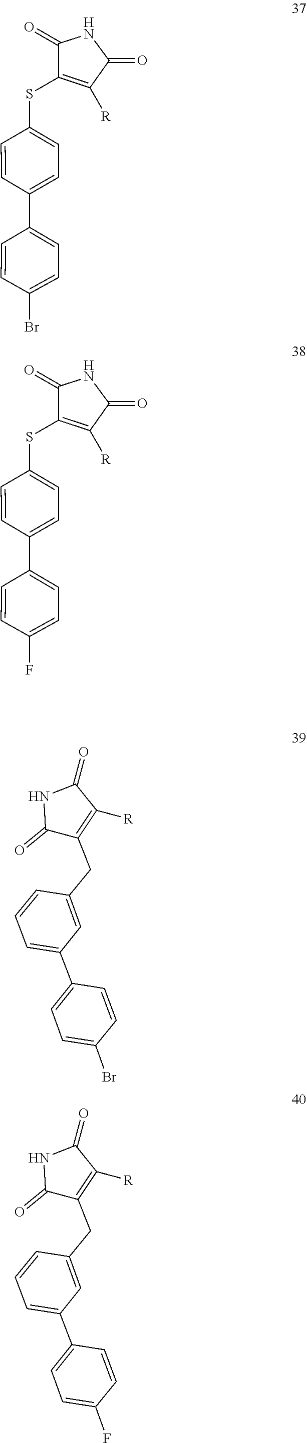

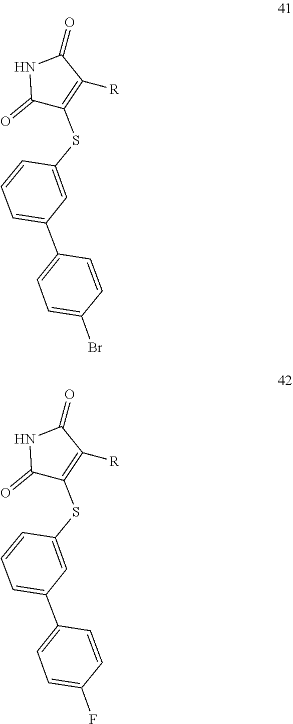

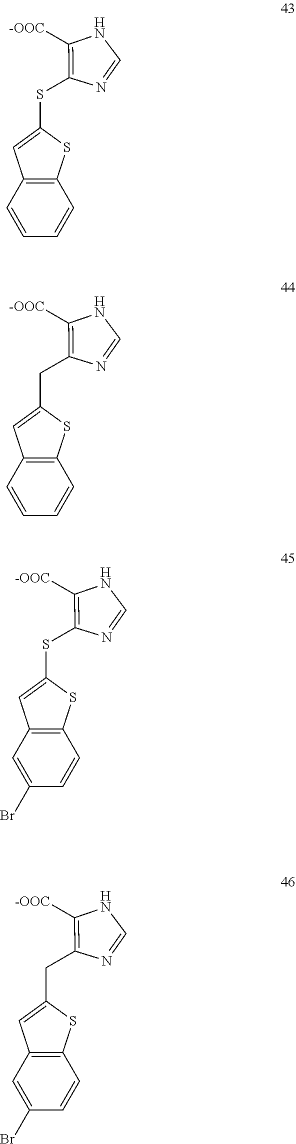

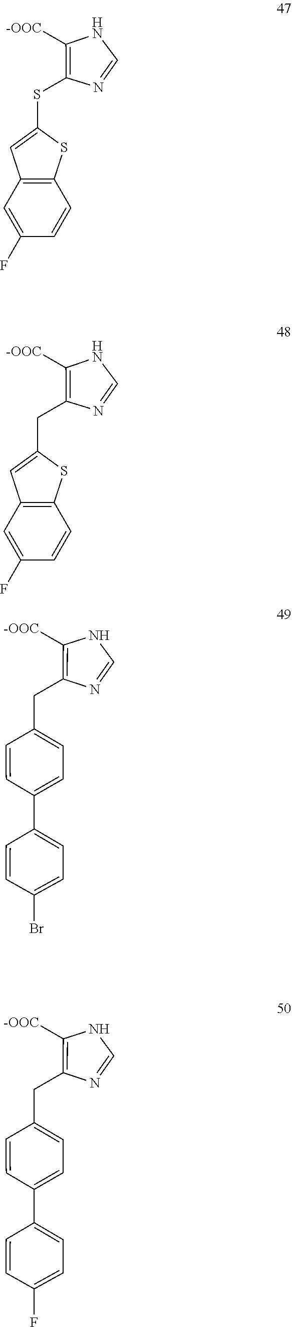

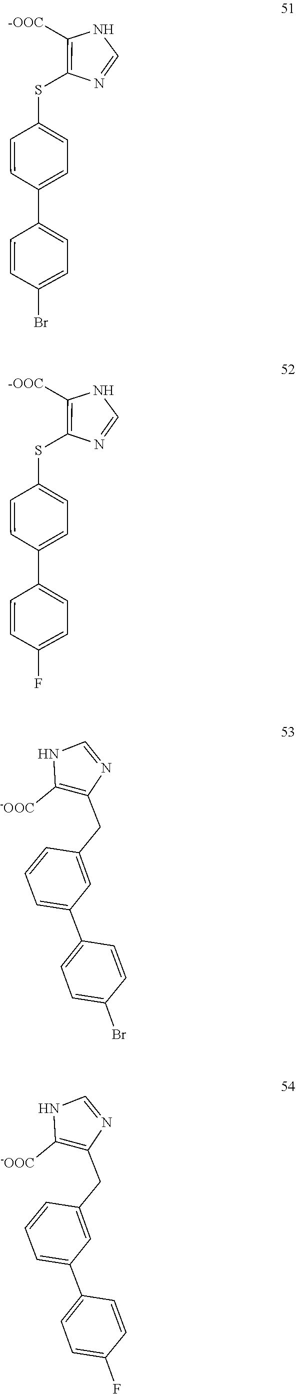

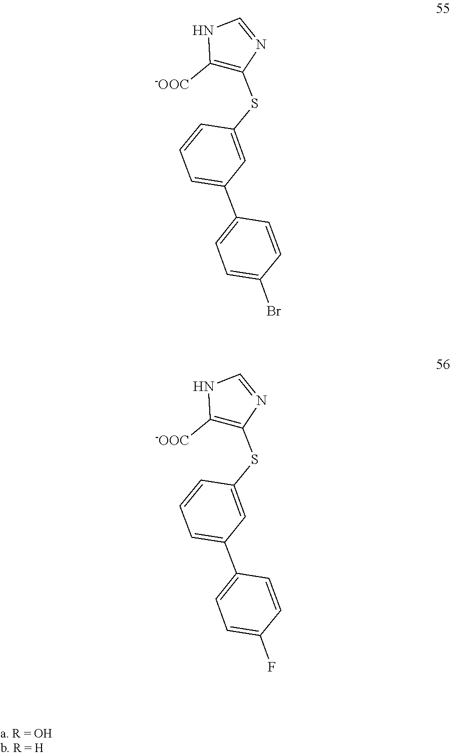

Example 3. Testing of inhibitors of GO. The inhibition of recombinant, human liver GO (the HAO1 gene product) is readily determined by a coupled assay that contains 2,6-dichloroindophenol (DCIP) (Murray et al., Biochemistry 47, 2439-2449, 2008). Briefly, GO is pre-incubated at 37 ° C. with or without inhibitor in 100 mM potassium phosphate pH 7.5 (0.1% DMSO final) for 5 min. An aliquot of pre-warmed DCIP and glycolate is added to start the reaction (final concentration 75 μM DCIP, 3 mM glycolate). The reaction rate is determined by monitoring the decrease at 600 nm (extinction coefficient of 21 mM⁻¹ cm ⁻¹). CDST inhibits GO with an apparent Ki of ˜15 nM.

Example 4. Therapy with GO inhibitor. Subjects are administered a GO inhibitor to treat kidney stones.

### Literature Cited

Adams, E., and Frank, L. (1980) Metabolism of proline and the hydroxyprolines. Annu Rev Biochem 49, 1005-1061

Belostotsky, R., Pitt, J. J., and Frishberg, Y. (2012) Primary hyperoxaluria type III--a model for studying perturbations in glyoxylate metabolism. J Mol Med (Berl) 90, 1497-1504

Atlante, A., Passarella, S., and Quagliariello, E. (1994) Spectroscopic study of hydroxyproline transport in rat kidney mitochondria. Biochem Biophys Res Commun 202, 58-64

Curhan, G. C., and Taylor, E. N. (2008) 24-h uric acid excretion and the risk of kidney stones. Kidney Int 73, 489-496

Efron, M. L., Bixby, E. M., and Pryles, C. V. (1965) Hydroxyprolinemia. Ii. A Rare Metabolic Disease Due to a Deficiency of the Enzyme “Hydroxyproline Oxidase”. New Engl J Med 272, 1299-1309

Frishberg, Y., Zeharia, A., Lyakhovetsky, R. Bargal, R. and Belostotsky, R. (2014) Mutations in Hao1 encoding glycolate oxidase cause isolated glycoli aciduria. J Med Genet 51, 526-529

Murray, M.S., Holmes, R.P. and Lowther, W. T. (2008) Active site loop 4 movements with human glycoalte oxidase: implications for substrate specificity and drug design. Biochemistry 47, 2439-2449.

Jiang, J., Johnson, L. C., Knight, J., Callahan, M. F., Riedel, T. J., Holmes, R. P., and Lowther, W. T. (2012) Metabolism of [13C5]hydroxyproline in vitro and in vivo: implications for primary hyperoxaluria. Am J Physiol Gastrointest Liver Physiol 302, G637-643

Jones, J. M, Morrell, J.C. and Gould, S. J. (2000) Indentification and characterization of HAOX1, HOAX2, and HOAX3, three human peroxisome 2-hydorxy acid oxidases. J Biol Chem 275, 12590-12597

Khan, S. R., and Glenton, P. A. (2010) Experimental induction of calcium oxalate nephrolithiasis in mice. J Urol 184, 1189-1196

Khan, S. R., Glenton, P. A., and Byer, K. J. (2006) Modeling of hyperoxaluric calcium oxalate nephrolithiasis: experimental induction of hyperoxaluria by hydroxy-L-proline. Kidney Int 70, 914-923

Kivirikko, K. I. (1970) Urinary excretion of hydroxyproline in health and disease. Int Rev Connect Tissue Res 5, 93-163

Knight, J., and Holmes, R. P. (2005) Mitochondrial hydroxyproline metabolism: implications for primary hyperoxaluria. Am J Nephrol 25, 171-175

Knight, J., Holmes, R. P., Cramer, S. D., Takayama, T., and Salido, E. (2012) Hydroxyproline metabolism in mouse models of primary hyperoxaluria. Am J Physiol-Renal 302, F688-693

Knight, J., Jiang, J., Assimos, D. G., and Holmes, R. P. (2006) Hydroxyproline ingestion and urinary oxalate and glycolate excretion. Kidney Int 70, 1929-1934

Lipinski, C. A., Lombardo, F., Dominy, B. W., and Feeney, P. J. (2001) Experimental and computational approaches to estimate solubility and permeability in drug discovery and development settings. Adv Drug Deliv Rev 46, 3-26

Miyata, N., Steffen, J., Johnson, M. E., Fargue, S., Danpure, C. J., and Koehler, C. M. (2014) Pharmacologic rescue of an enzyme-trafficking defect in primary hyperoxaluria 1. Proc Natl Acad Sci USA 111, 14406-14411

Monico, C. G., Rossetti, S., Belostotsky, R., Cogal, A. G., Herges, R. M., Seide, B. M., Olson, J. B., Bergstrahl, E. J., Williams, H. J., Haley, W. E., Frishberg, Y., and Milliner, D. S. (2011) Primary hyperoxaluria type III gene HOGA1 (formerly DHDPSL) as a possible risk factor for idiopathic calcium oxalate urolithiasis. Clin J Am Soc Nepthrol 6, 2289-2295

Moxley, M. A., and Becker, D. F. (2012) Rapid reaction kinetics of proline dehydrogenase in the multifunctional proline utilization A protein. Biochemistry 51, 511-520

Moxley, M. A., Tanner, J. J., and Becker, D. F. (2011) Steady-state kinetic mechanism of the proline:ubiquinone oxidoreductase activity of proline utilization A (PutA) from Escherichia coli. Arch Biochem Biophys 516, 113-120

Ostrander, E. L., Larson, J. D., Schuermann, J. P., and Tanner, J. J. (2009) A Conserved Active Site Tyrosine Residue of Proline Dehydrogenase Helps Enforce the Preference for Proline over Hydroxyproline as the Substrate. Biochemistry 48, 951-959

Pelkonen, R., and Kivirikko, K. I. (1970) Hydroxyprolinemia: an apparently harmless familial metabolic disorder. New Engl J Med 283, 451-456

Pemberton, T. A., and Tanner, J. J. (2013) Structural basis of substrate selectivity of Delta(1)-pyrroline-5-carboxylate dehydrogenase (ALDH4A1): semialdehyde chain length. Arch Biochem Biophys 538, 34-40

Phang, J. M., Hu, C. A., and Valle, D. (2001) Disorders of proline and hydroxyproline metabolism. in The Metabolic and Molecular Bases of Inherited Disease (Scriver, C. R., Beaudet, A. L., Sly, W. S., Vallee, D., Childs, B., Kinzler, K. W., and Vogelstein, B. eds.), McGraw-Hill, New York. pp 1821-1838

Riedel, T. J., Johnson, L. C., Knight, J., Hantgan, R. R., Holmes, R. P., and Lowther, W. T. (2011) Structural and Biochemical Studies of Human 4-hydroxy-2-oxoglutarate Aldolase: Implications for Hydroxyproline Metabolism in Primary Hyperoxaluria. PLoS One 6, e26021

Riedel, T. J., Knight, J., Murray, M. S., Milliner, D. S., Holmes, R. P., and Lowther, W. T. (2012) 4-Hydroxy-2-oxoglutarate aldolase inactivity in primary hyperoxaluria type 3 and glyoxylate reductase inhibition. Biochim Biophys Acta 1822, 1544-1552

Roy, A., Kucukural, A., and Zhang, Y. (2010) I-TASSER: a unified platform for automated protein structure and function prediction. Nature Protoc 5, 725-738

Srivastava, D., Schuermann, J P , White, T. A., Krishnan, N., Sanyal, N., Hura, G. L., Tan, A., Henzl, M. T., Becker, D. F., and Tanner, J. J. (2010) Crystal structure of the bifunctional proline utilization A flavoenzyme from Bradyrhizobium japonicum. Proceedings of the National Academy of Sciences of the United States of America 107, 2878-2883

Tallarita, E., Pollegioni, L., Servi, S., and Molla, G. (2012) Expression in Escherichia coli of the catalytic domain of human proline oxidase. Protein Expres Purif 82, 345-351

Valle, D., Goodman, S. I., Harris, S. C., and Phang, J. M. (1979) Genetic evidence for a common enzyme catalyzing the second step in the degradation of proline and hydroxyproline. J Clin Invest 64, 1365-1370

White, T. A., Krishnan, N., Becker, D. F., and Tanner, J. J. (2007) Structure and kinetics of monofunctional proline dehydrogenase from Thermus thermophilus. J Biol Chem 282, 14316-14327

Williams, I., and Frank, L. (1975) Improved chemical synthesis and enzymatic assay of delta-1-pyrroline-5-carboxylic acid. Anal Biochem 64, 85-97

Williams, H. J., Williams, N., Spurlock, G., Norton, N., Zammit, S., Kirov, G., Owen, M. J., and O' Donovan, M. C. (2003) Detailed analysis of PRODH and PsPRODH reveals no association with schizophrenia. Am J Med Genet B Neuropsychiatr Genet 120B, 42-46

Willis, A., Bender, H. U., Steel, G., and Valle, D. (2008) PRODH variants and risk for schizophrenia. Amino Acids 35, 673-679

Wold, J. P., Lundby, F., and Egelandsdel, B. (1999) Quantification of connective tissue (hydroxyproline) in ground beef by autofluorescence spectroscopy. J Food Sc 64, 377-383

Zhang, Y. (2008) I-TASSER server for protein 3D structure prediction. BMC Bioinformatics 9, 40

Example 5. Mouse model. Mice that do not express GO have been generated. The Hao1

(GO) deficient animals developed normally and exhibited similar behavior to wild-type litter mates. The genotype of each mouse was confirmed by PCR analysis from a tail snip. Liver was analyzed by western analysis. These tests confirmed that the Hao1 homozygous mouse did not contain GO in any of the samples. As expected, GO is not present in the kidney of all mouse strains.

Mice lacking GO appear normal apart from an increased urinary glycolate excretion and elevated plasma glycolate level. Male mice lacking GO excreted ˜1.4 fold more urinary oxalate than wild type litter mates; however, female Hao1 deficient mice show no significant difference in urinary oxalate excretion compared to Wt litter mates. It is noted that this finding with male Hao1 deficient mice is not consistent with data recently published by Dr. Salido's group that showed no difference in urinary oxalate excretion between Wt and Hao1 deficient male mice. However, the diet used by Dr. Salido's group was different from that used in this study.

The heterozygous (Htz) Hao1 mouse strain showed reduced expression of protein as measured by western Blot.

Given the cycle of glycolate-glyoxylate interconversion that will occur via GO and glyoxylate reductase activities in hepatocytes lacking alanine-glyoxylate aminotransferase (AGT), inhibition of GO is likely to reduce oxalate synthesis in PH1 patients. The contribution of glycolate to oxalate synthesis in humans with functional AGT activity is not known; however, individuals lacking GO appear normal. Frishberg et al., J Med Genet 51(8):526-9 (2014).Therefore, strategies to reduce GO activity may provide benefit for reducing urinary oxalate excretion in patients with calcium oxalate kidney stone disease.

The foregoing is illustrative of the present invention, and is not to be construed as limiting thereof. The invention is defined by the following claims, with equivalents of the claims to be included therein.

1. A compound of Formula I: 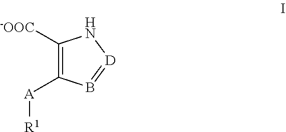
wherein:
A is CH₂ or S;
B is CH or N;
D is CH or N; and
R¹ is aryl or heteroaryl, wherein said aryl or heteroaryl has two aromatic rings, which rings are fused or directly adjoining,
or a pharmaceutically acceptable salt or prodrug thereof.

2.-7. (canceled)

8. The compound of claim 1, wherein R¹ is benzothiophene or biphenyl.

9. The compound of claim 1, wherein R¹ is selected from the group consisting of: 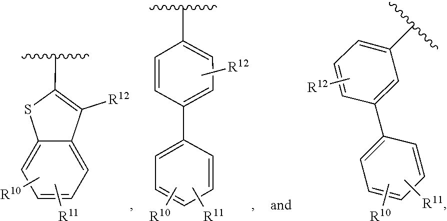
wherein R¹⁰, R¹¹ and R¹² are each independently selected from the group consisting of: H, alkyl, halo and haloalkyl,
or a pharmaceutically acceptable salt or prodrug thereof.

10. The compound of claim 1, wherein said compound is selected from the group consisting of: 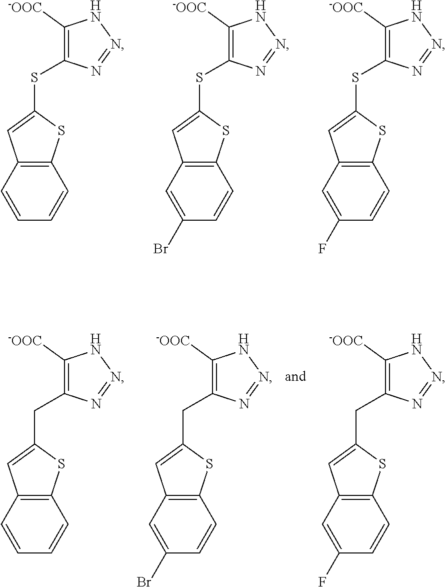
or a pharmaceutically acceptable salt thereof.

11. The compound of claim 1, wherein said compound is selected from the group consisting of: 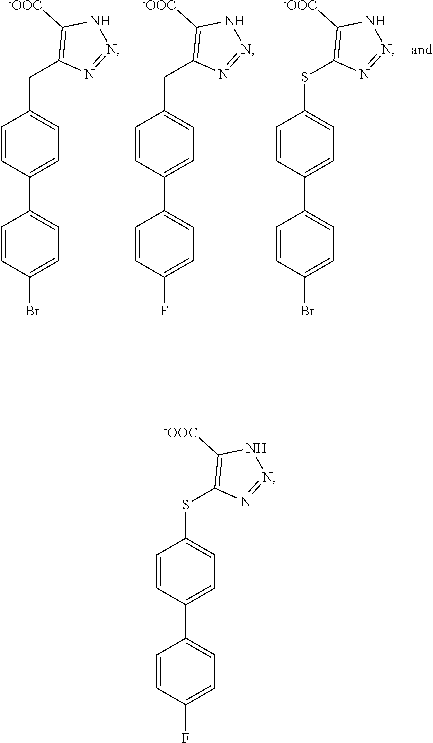
or a pharmaceutically acceptable salt thereof.

12. The compound of claim 1, wherein said compound is selected from the group consisting of: 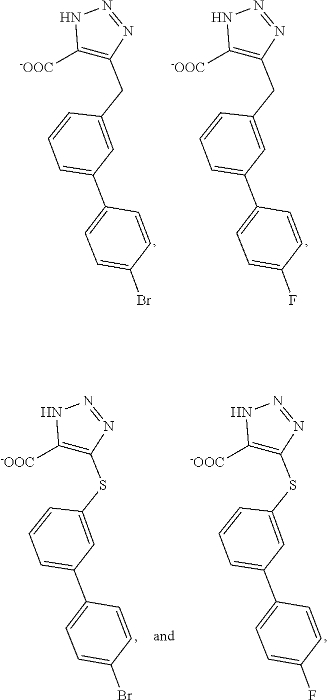
or a pharmaceutically acceptable salt thereof.

13. compound of claim 1, wherein said compound is selected from the group consisting of: 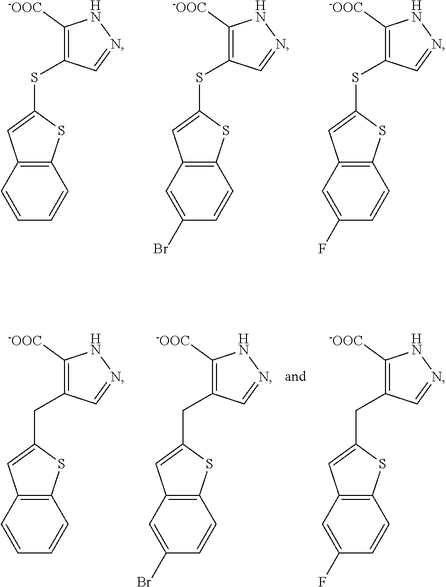
or a pharmaceutically acceptable salt thereof.

14. The compound of claim 1, wherein said compound is selected from the group consisting of: 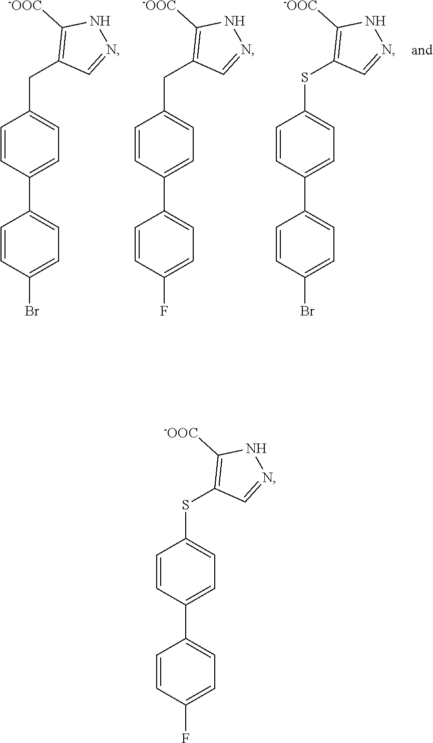
or a pharmaceutically acceptable salt thereof.

15. The compound of claim 1, wherein said compound is selected from the group consisting of: 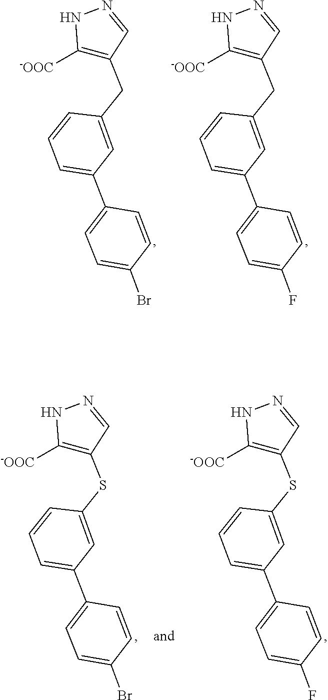
or a pharmaceutically acceptable salt thereof.

16. The compound of claim 1, wherein said compound is selected from the group consisting of: 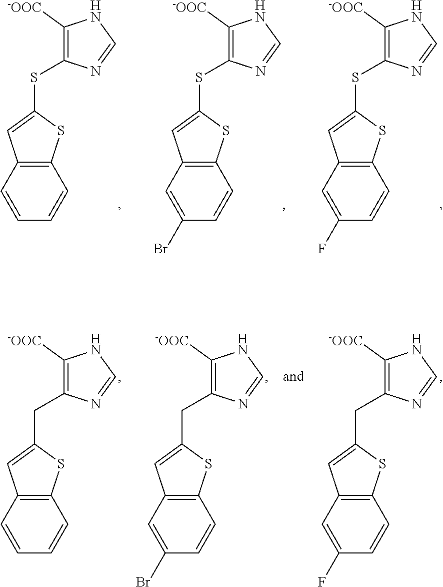
or a pharmaceutically acceptable salt thereof.

17. The compound of claim 1, wherein said compound is selected from the group consisting of: 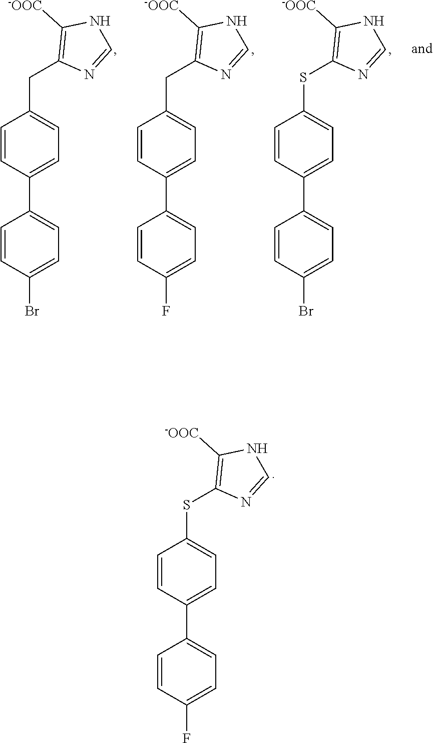
or a pharmaceutically acceptable salt thereof.

18. The compound of claim 1, wherein said compound is selected from the group consisting of: 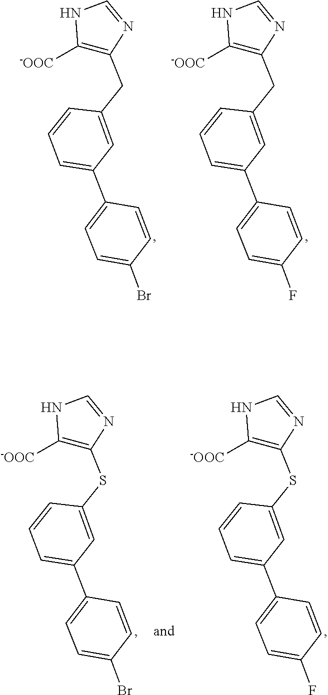
or a pharmaceutically acceptable salt thereof.

19. A compound of Formula II: 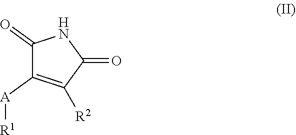
wherein:
A is CH₂ or S;
R¹ is aryl or heteroaryl, wherein said aryl or heteroaryl has two aromatic rings, which rings are fused or directly adjoining; and
R² is H or OH,
or a pharmaceutically acceptable salt or prodrug thereof.

20.-23. (canceled)

24. The compound of claim 19, wherein R¹ is benzothiophene or biphenyl.

25. The compound of, wherein R¹ is selected from the group consisting of: 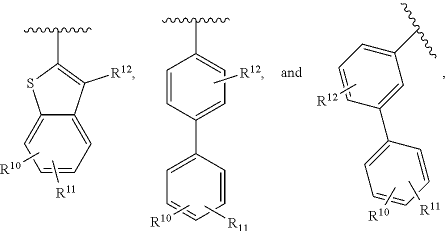
wherein R¹⁰, R¹¹ and R¹² are each independently selected from the group consisting of: H, alkyl, halo and haloalkyl,
or a pharmaceutically acceptable salt or prodrug thereof.

26. The compound of claim 19, wherein said compound is selected from the group consisting of: 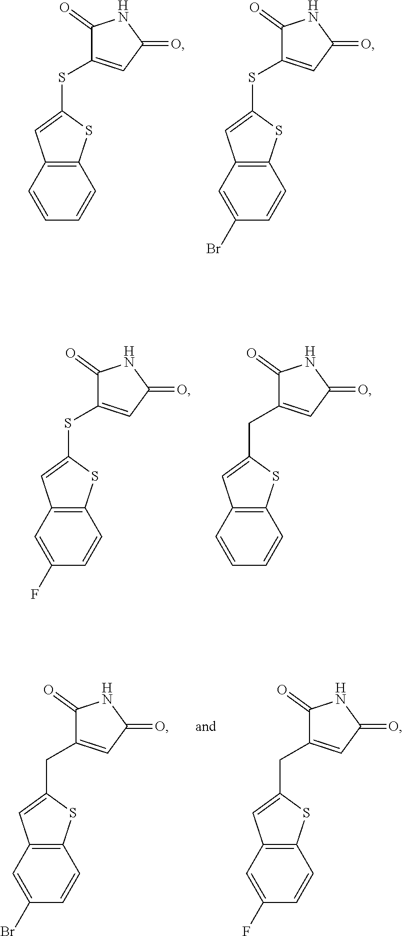 [SMILES: O=C1C=C(Sc2cc3ccccc3s2)C(=O)N1 ; O=C1C=C(Sc2cc3cc(Br)ccc3s2)C(=O)N1 ; O=C1C=C(Sc2cc3cc(F)ccc3s2)C(=O)N1 ; O=C1C=C(Cc2cc3ccccc3s2)C(=O)N1 ; O=C1C=C(Cc2cc3cc(Br)ccc3s2)C(=O)N1 ; O=C1C=C(Cc2cc3cc(F)ccc3s2)C(=O)N1]
or a pharmaceutically acceptable salt thereof.

27. The compound of claim 19, wherein said compound is selected from the group consisting of: 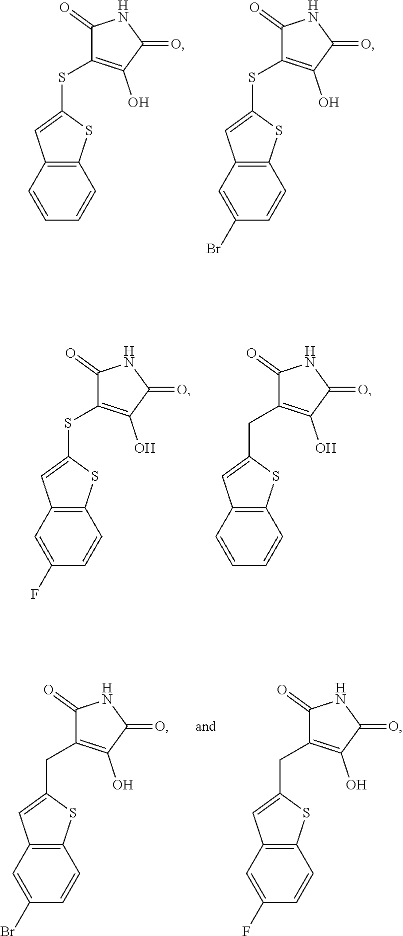 [SMILES: O=C1NC(=O)C(Sc2cc3ccccc3s2)=C1O ; O=C1NC(=O)C(Sc2cc3cc(Br)ccc3s2)=C1O ; O=C1NC(=O)C(Sc2cc3cc(F)ccc3s2)=C1O ; O=C1NC(=O)C(Cc2cc3ccccc3s2)=C1O ; O=C1NC(=O)C(Cc2cc3cc(Br)ccc3s2)=C1O ; O=C1NC(=O)C(Cc2cc3cc(F)ccc3s2)=C1O]
or a pharmaceutically acceptable salt thereof.

28. The compound of claim 19, wherein said compound is selected from the group consisting of: 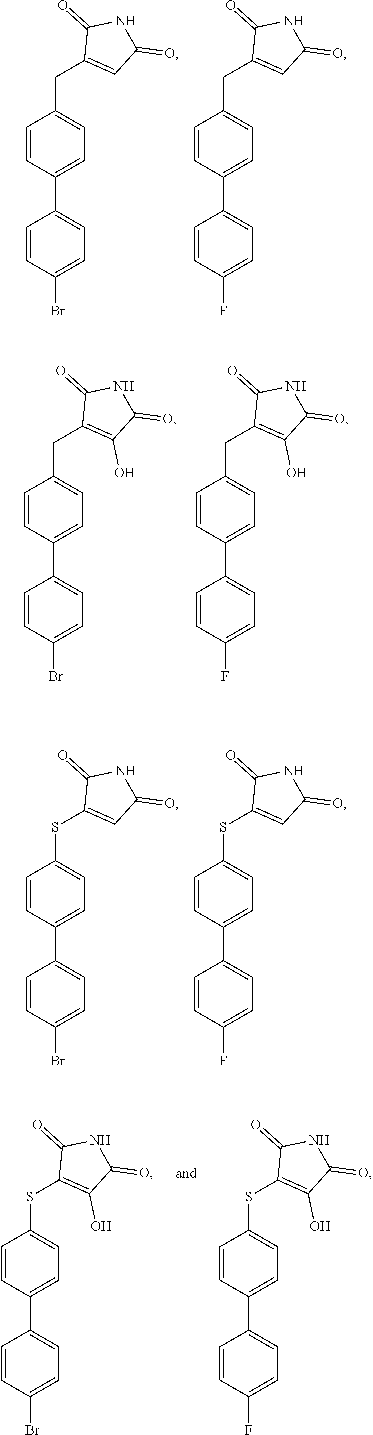
or a pharmaceutically acceptable salt thereof.

29. The compound of claim 19, wherein said compound is selected from the group consisting of: 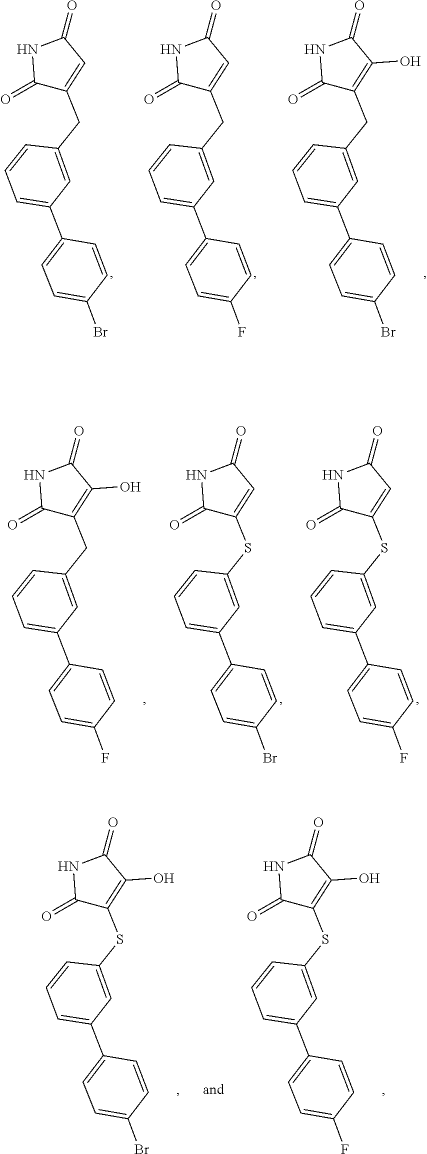
or a pharmaceutically acceptable salt thereof.

30. A pharmaceutical composition comprising a compound, pharmaceutically acceptable salt or prodrug of claim 1.

31. The pharmaceutical composition of claim 30, wherein said composition is formulated for oral administration.

32. The pharmaceutical composition of claim 30, wherein said composition is a food product formulation.

33. The pharmaceutical composition of claim 30, wherein said composition is a capsule, cachet, lozenge, or tablet.

34. The pharmaceutical composition of claim 30, wherein said formulation is provided in unit dosage form of from 1 mg to 10 grams of the compound, pharmaceutically acceptable salt or prodrug.

35. A method of treating kidney stones, comprising: administering to a subject in need thereof a therapeutically effective amount of the compound, pharmaceutically acceptable salt or prodrug of claim 1.

36. A method of inhibiting the production of glyoxylate and/or oxalate, and/or inhibiting glycolate oxidase (GO), in a subject in need thereof, comprising:
administering to said subject a therapeutically effective amount of the compound, pharmaceutically acceptable salt or prodrug of claim 1.

37. The method of claim 35, wherein said subject is a human subject.

38. The method of claim 35, wherein said subject is a non-human animal subject.

39.-40. (canceled)
